(19) United States
(12) Patent Application Publication
(10) Pub. No.: US 2011/0021515 A1
(43) Pub. Date: Jan. 27, 2011

(54) DIHYROFUROPYRMINDINE COMPOUNDS

(75) Inventors: Qing DONG, San Diego, CA (US); Bohan Jin, San Diego, CA (US); Matthew Lardy, San Diego, CA (US); Feng Zhao, Escondido, CA (US)

Correspondence Address: TAKEDA SAN DIEGO, INC., 10410 SCIENCE CENTER DRIVE, SAN DIEGO, CA, 92121, US

(73) Assignee: TAKEDA PHARMACEUTICAL COMPANY LIMITED, Osaka (JP)

(21) Appl. No.: 12/841,940

(22) Filed: Jul. 22, 2010

Related U.S. Application Data
(60) Provisional application No. 61/228,428, filed on Jul. 24, 2009.

(51) Int. Cl. A61K 31/5377; C07D 491/048; C07D 498/08; A61K 31/5386; A61P 35/00; A61P 37/06

(52) U.S. Cl. 514/230.5; 544/117; 514/234.2; 544/105; 544/70

(57) ABSTRACT

The present invention provides mTOR inhibitors of the formula

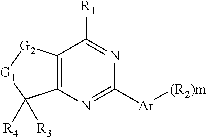

wherein the variables are as defined herein. Also provided are pharmaceutical compositions, kits and articles of manufacture comprising such compounds; methods of making the compounds and intermediates thereof; and methods of using the compounds.

### RELATED APPLICATION

This application claims the benefit of U.S. Provisional Application No. 61/228,428, filed Jul. 24, 2009, which is incorporated herein by reference.

### FIELD OF THE INVENTION

The present invention relates to medicinal chemistry and pharmaceutical science. Provided herein are compounds that inhibit mammalian target of rapamycin (mTOR).

### BACKGROUND OF THE INVENTION

mTOR is a serine/threonine kinase and has been identified as a regulator of protein synthesis as well as cell growth and proliferation. Also, mTOR has been shown to regulate the response of tumor cells to nutrients and growth factors as well as the ability of tumors to promote angiogenesis. Thus, inhibitors of mTOR activity are being actively studied as potential anti-proliferative agents. Currently inhibitors of mTOR are approved for immunosuppression and cancer treatment.

Inhibition of mTOR function by small molecules results in a loss of transmission of upstream activating signals (i.e., from growth factor receptors) to downstream effectors of cell growth. Rapamycin, an inhibitor of mTOR, inhibits proliferation or growth of cells derived from a range of tissue types such as smooth muscle and T-cells as well as cells derived from a diverse range of tumor types including rhabdomyosarcoma, neuroblastoma, glioblastoma and medulloblastoma, small cell lung cancer, osteosarcoma, pancreatic carcinoma and breast and prostate carcinoma. Moreover, Rapamycin and its derivatives have shown the ability to potentiate the cytotoxicity of a number of common cancer chemotherapies including cisplatin, camptothecin and doxorubicin.

It has been shown that mTOR functions in two distinct complexes (mTORC1 and mTORC2). Rapamycin primarily inhibits the mTORC1 complex while largely sparing mTORC2 activity. Thus, one strategy is to identify compounds that are capable of inhibiting mTORC1 and mTORC2 mediated activity in the cell. The compounds of the present invention are such inhibitors of mTOR and are useful to treat disorders associated with mTOR.

Certain inhibitors of mTOR are disclosed in WO 2008/023180 and WO 2007/060404. Certain inhibitors of mTOR and/or PI3K are disclosed in WO 2008/023180, WO 2008/115974, and WO 2009/052145. Certain inhibitors of PI3K are disclosed in WO 2008/152394. Certain inhibitors of voltage-gated sodium channels are disclosed in WO 2005/014558. Certain modulators of the ABC transporter are disclosed in WO 2004/111014. Certain insulin secretion promotors are disclosed in US 2008/0070896.

### SUMMARY OF THE INVENTION

The present invention provides compounds of formula I:

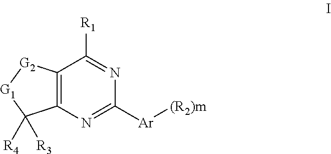

Ar is selected from the group consisting of C₄₋₁₄ aryl, and C₁₋₁₀ heteroaryl;
R₁ is selected from the group consisting of optionally substituted C₃₋₈ cycloalkyl, and optionally substituted C₃₋₁₂ heterocycloalkyl;
R₂ is, each time taken, independently selected from the group consisting of halo, cyano, optionally substituted C₁₋₆ alkyl, C₁₋₈ sulfonyl, optionally substituted C₂₋₄ alkenyl, optionally substituted C₂₋₄ alkynyl, optionally substituted C₁₋₄ alkoxy, C₀₋₈ alkylamino, optionally substituted C₄₋₁₄ aryl, optionally substituted C₄₋₁₄ aryloxy, C₁₋₅ oxycarbonyl, C₁₋₅ carbonyloxy, optionally substituted C₃₋₆ heterocycloalkyl, optionally substituted C₁₋₁₀ heteroaryl, hydroxy, nitro, —NHC(O)NR₇R₈, —NHC(O)OR₉, —NH(SO₂)NHR₇, —NHC(O)NHNR₇R₈, —NHC(S)NR₇R₈, —NHC(═NR₁₀)NR₇R₈, —NHC(SR₁₁)NR₇R₈, and —NHC(═NR₁₀)OR₁₂;
R₃ is selected from the group consisting of halo, optionally substituted C₁₋₆ alkyl, C₁₋₈ sulfonyl, optionally substituted C₂₋₄ alkenyl, optionally substituted C₁₋₄ alkoxy, and optionally substituted C₃₋₈ cycloalkyl;
R₄ is selected from the group consisting of halo, optionally substituted C₁₋₆ alkyl, C₁₋₈ sulfonyl, optionally substituted C₂₋₄ alkenyl, optionally substituted C₁₋₄ alkoxy, and optionally substituted C₃₋₈ cycloalkyl; or
R₃ and R₄ are taken together along with the carbon to which they are attached to form a spiro mono- or bi-cyclic ring having 3 to 10 carbon atoms and optionally having 1, 2, or 3 heteroatoms independently selected from the group consisting of oxygen, nitrogen and sulfur and optionally oxidized on sulfur to provide the sulfoxides and sulfone and optionally substituted on the ring carbons with 1 to 4 substituents, each time taken, independently selected from the group consisting of optionally substituted C₁₋₄ alkyl, C₂₋₄ alkenyl, C₁₋₄ alkoxy, C₁₋₉ amide, C₁₋₇ amido, C₀₋₈ alkylamino, C₁₋₅ oxycarbonyl, cyano, C₃₋₈ cycloalkyl, C₃₋₈ cycloalkoxy, halo, hydroxy, nitro, oxo, and optionally substituted phenyl, and optionally substituted on the ring nitrogens, each time taken, with a substituent selected from the group consisting of optionally substituted C₁₋₄ alkyl, C₂₋₄ alkenyl, C₃₋₈ cycloalkyl, optionally substituted C₃₋₆ heterocycloalkyl, and optionally substituted phenyl;
G₁ is selected from the group consisting of O and CR₅R₆;
G₂ is selected from the group consisting of O and CR₅R₆;
provided that one of G₁ or G₂ is O and the other is CR₅R₆;
R₅ is selected from the group consisting of hydrogen, halo, optionally substituted C₁₋₆ alkyl, optionally substituted C₂₋₄ alkenyl, and optionally substituted C₃₋₈ cycloalkyl;
R₆ is selected from the group consisting of hydrogen, halo, optionally substituted C₁₋₆ alkyl, optionally substituted C₂₋₄ alkenyl, and optionally substituted C₃₋₈ cycloalkyl;
R₇ is, each time taken, independently selected from the group consisting of hydrogen, optionally substituted C₁₋₆ alkyl, optionally substituted C₃₋₈ cycloalkyl, optionally substituted C₄₋₁₄ aryl, optionally substituted C₃₋₆ heterocycloalkyl, and optionally substituted C₁₋₁₀ heteroaryl;
R₈ is, each time taken, independently selected from the group consisting of hydrogen, optionally substituted C₁₋₆ alkyl, optionally substituted C₃₋₈ cycloalkyl, optionally substituted C₄₋₁₄ aryl, optionally substituted C₃₋₆ heterocycloalkyl, and optionally substituted C₁₋₁₀ heteroaryl;
R₉ is, each time taken, independently selected from the group consisting of optionally substituted C₁₋₆ alkyl, optionally substituted C₃₋₈ cycloalkyl, optionally substituted C₄₋₁₄ aryl, and optionally substituted C₃₋₆ heterocycloalkyl;
R₁₀ is, each time taken, independently selected from the group consisting of hydrogen, optionally substituted C₁₋₆ alkyl, optionally substituted C₁₋₆ alkoxy, optionally substituted C₃₋₈ cycloalkyl, optionally substituted C₄₋₁₄ aryl, optionally substituted C₃₋₆ heterocycloalkyl, optionally substituted C₁₋₁₀ heteroaryl, cyano, and nitro;
R₁₁ is each time taken independently selected from the group consisting of optionally substituted C₁₋₆ alkyl and optionally substituted phenyl;
R₁₂ is each time taken independently selected from the group consisting of optionally substituted C₁₋₆ alkyl, optionally substituted C₃₋₈ cycloalkyl, and optionally substituted C₄₋₁₄ aryl;
m is 0, 1, 2, 3, and 4;
or the pharmaceutically acceptable salts thereof.

The present invention also provides pharmaceutical compositions, comprising: a compound of formula I and a pharmaceutically acceptable excipient.

The compounds of the invention are inhibitors of mTOR they are useful for the treatment of conditions associated with mTOR, including cancer. Thus, the invention provides methods of treating conditions associated with mTOR, comprising: administering to a patient in need thereof an effective amount of a compound of formula I. Further, the present invention provides for the use of compounds of formula I, including for the manufacture of a medicament, each specifically including for the treatment of particular conditions associated with mTOR.

The present invention also provides an article of manufacture: comprising at least one compound of formula I and a label. Also provided are kits comprising at least one compound of the invention, a label, and apparatus for administration of the inhibitor.

The present invention also provides processes from making mTOR inhibitors and intermediates thereof.

### DETAILED DESCRIPTION OF THE INVENTION

The term “C₂₋₄ alkenyl” refers to a straight or branched alkenyl chain having from two to four carbon atoms and one or more carbon-carbon double bonds, and includes ethylene, propylene, iso-propylene, butylene, iso-butylene, sec-butylene, and the like.

The term “optionally substituted C₂₋₄ alkenyl” refers to a C₂₋₄ alkenyl optionally having from 1 to 3 substituents independently selected from the group consisting of C₁₋₄ alkoxy, C₁₋₉ amide, C₁₋₅ oxycarbonyl, cyano, C₃₋₈ cycloalkyl, halo, hydroxy, optionally substituted C₁₋₁₀ heteroaryl, and optionally substituted phenyl.

The term “C₁₋₄ alkyl” refers to a straight or branched alkyl chain having from one to four carbon atoms.

The term “optionally substituted C₁₋₄ alkyl” refers to a C₁₋₄ alkyl optionally having from 1 to 5 substituents independently selected from the group consisting of C₂₋₄ alkenyl, C₁₋₄ alkoxy, C₁₋₄ thioalkoxy, C₁₋₉ amide, C₀₋₈ alkylamino, C₁₋₅ oxycarbonyl, cyano, C₃₋₈ cycloalkyl, C₃₋₈ cycloalkoxy, halo, hydroxy, nitro, oxo, optionally substituted C₃₋₆ heterocycloalkyl, optionally substituted C₁₋₁₀ heteroaryl, and optionally substituted phenyl. A particular “optionally substituted C₁₋₄ alkyl” is one optionally having from 1 to 4 substituents independently selected from the group consisting of C₀₋₈ alkylamino, C₁₋₄ alkoxy, C₁₋₉ amide, C₁₋₅ oxycarbonyl, cyano, C₃₋₈ cycloalkyl, halo, hydroxy, optionally substituted C₃₋₆ heterocycloalkyl, and optionally substituted phenyl.

The term “C₁₋₆ alkyl” refers to a straight or branched alkyl chain having from one to six carbon atoms.

The term “optionally substituted C₁₋₆ alkyl” refers to a C₁₋₆ alkyl optionally having from 1 to 7 substituents independently selected from the group consisting of C₀₋₈ alkylamino, C₂₋₄ alkenyl, C₁₋₄ alkoxy, C₁₋₄ thioalkoxy, C₁₋₉ amide, C₁₋₅ oxycarbonyl, cyano, C₃₋₈ cycloalkyl, halo, hydroxy, optionally substituted C₁₋₁₀ heteroaryl, optionally substituted C₃₋₆ heterocycloalkyl, and optionally substituted phenyl. A particular “optionally substituted C₁₋₆ alkyl” is one optionally having from 1 to 5 substituents independently selected from the group consisting of C₀₋₈ alkylamino, C₁₋₄ alkoxy, C₁₋₉ amide, C₁₋₅ oxycarbonyl, cyano, C₃₋₈ cycloalkyl, halo, hydroxy, optionally substituted C₃₋₆ heterocycloalkyl, and optionally substituted phenyl.

The term “C₁₋₈ sulfonyl” refers to a sulfonyl linked to a C₁₋₆ alkyl group, C₃₋₈ cycloalkyl, or an optionally substituted phenyl.

The term “C₁₋₄ alkoxy” refers to a C₁₋₄ alkyl attached through an oxygen atom.

The term “optionally substituted C₁₋₄ alkoxy” refers to a C₁₋₄ alkoxy optionally having from 1 to 6 substituents independently selected from the group consisting of C₂₋₄ alkenyl, C₁₋₄ alkoxy, C₁₋₉ amide, C₁₋₅ oxycarbonyl, cyano, C₃₋₈ cycloalkyl, halo, hydroxy, optionally substituted C₁₋₁₀ heteroaryl, and optionally substituted phenyl. While it is understood that where the optional substituent is C₁₋₄ alkoxy, cyano, halo, or hydroxy then the substituent is generally not alpha to the alkoxy attachment point, the term “optionally substituted C₁₋₄ alkoxy” specifically includes cyanomethoxy, trifluoromethoxy, difluoromethoxy, fluoromethoxy, and other stable moieties.

The term “C₂₋₄ alkynyl” refers to a straight or branched alkynyl chain having from two to four carbon atoms and one or more carbon-carbon triple bonds.

The term “optionally substituted C₂₋₄ alkynyl” refers to a C₂₋₆ alkynyl optionally having from 1 to 3 substituents independently selected from the group consisting of C₁₋₄ alkoxy, C₁₋₉ amide, C₁₋₅ oxycarbonyl, cyano, C₃₋₈ cycloalkyl, halo, hydroxy, optionally substituted C₁₋₁₀ heteroaryl, and optionally substituted phenyl.

The term “C₁₋₉ amide” refers to an amide having two groups independently selected from the group consisting of hydrogen and C₁₋₄ alkyl, for example, —CONH₂, —CONHCH₃, and —CON(CH₃)₂.

The term “C₁₋₇ amido” refers to a —NHC(O)R group in which R is C₁₋₆ alkyl.

The term “C₀₋₈ alkylamino” refers to an amino optionally having one or two C₁₋₄ alkyl.

The term “C₄₋₁₄ aryl” refers to a monocyclic and polycyclic unsaturated, conjugated hydrocarbon having aromatic character and having four to fourteen carbon atoms, and includes phenyl, biphenyl, indenyl, cyclopentyldienyl, fluorenyl, and naphthyl. Particular C₄₋₁₄ aryls are phenyl and naphthyl. A more particular C₄₋₁₄ aryl is phenyl.

The term “optionally substituted C₄₋₁₄ aryl” refers to a C₄₋₁₄ aryloptionally having 1 to 5 substituents independently selected from the group consisting of C₀₋₈ alkylamino, C₁₋₇ amido, C₁₋₉ amide, C₂₋₅ carbamoyl, C₁₋₆ sulfonylamido, C₀₋₆ sulfonylamino, C₁₋₅ ureido, optionally substituted C₁₋₄ alkyl, optionally substituted C₁₋₄ alkoxy, cyano, halo, hydroxyl, nitro, C₁₋₅ oxycarbonyl, and C₁₋₈ sulfonyl. A particular “optionally substituted C₄₋₁₄ aryl” is one optionally having 1 to 3 substituents independently selected from the group consisting of a C₁₋₄ alkyl, C₁₋₄ alkoxy, cyano, halo, hydroxyl, nitro, trifluoromethyl, and trifluoromethoxy. A more particular “optionally substituted C₄₋₁₄ aryl” is phenyl optionally having 1 to 3 substituents independently selected from the group consisting of a C₁₋₄ alkyl, C₁₋₄ alkoxy, cyano, halo, hydroxyl, nitro, trifluoromethyl, and trifluoromethoxy.

The term “C₄₋₁₄ aryloxy” refers to a C₄₋₁₄ aryl attached through an oxygen atom.

The term “optionally substituted C₄₋₁₄ aryloxy” refers to a C₄₋₁₄ aryloxy optionally having 1 to 5 substituents independently selected from the group consisting of C₀₋₈ alkylamino, C₁₋₄ alkyl, C₁₋₄ alkoxy, cyano, halo, hydroxyl, nitro, C₁₋₈ sulfonyl, and trifluoromethyl.

The term “C₁₋₅ oxycarbonyl” refers to a oxycarbonyl group (—CO₂H) and C₁₋₄ alkyl ester thereof.

The term “C₁₋₅ carbonyloxy” refers to a carbonyloxy group (—O₂CR) wherein R is C₁₋₄ alkyl, for example acetoxy.

The term “C₃₋₈ cycloalkyl” refers to an alkyl ring having from three to eight carbon atoms, and includes cyclopropyl, 2-methyl cyclopropyl, cyclobutyl, cyclopentyl, cyclohexyl, and the like.

The term “optionally substituted C₃₋₈ cycloalkyl” refers to a C₃₋₈ cycloalkyl optionally having from 1 to 6 substituents independently selected from the group consisting of optionally substituted C₁₋₄ alkyl, C₂₋₄ alkenyl, C₁₋₄ alkoxy, C₁₋₉ amide, C₁₋₇ amido, C₀₋₈ alkylamino, C₁₋₅ oxycarbonyl, cyano, C₃₋₈ cycloalkyl, C₃₋₈ cycloalkoxy, halo, hydroxy, nitro, oxo, optionally substituted C₁₋₁₀ heteroaryl, and optionally substituted phenyl. A particular “optionally substituted C₃₋₈ cycloalkyl” is one optionally having from 1 to 3 substituents independently selected from the group consisting of C₁₋₄ alkyl, C₁₋₄ alkoxy, C₀₋₈ alkylamino, C₁₋₅ oxycarbonyl, cyano, halo, and hydroxy.

The term “C₃₋₈ cycloalkoxy” refers to a C₃₋₈ cycloalkyl attached through an oxygen atom.

The terms “halogen” and “halo” refers to a chloro, fluoro, bromo or iodo atom.

The term “C₃₋₆ heterocycloalkyl” refers to a 4 to 10 membered monocyclic saturated or partially (but not fully) unsaturated ring having three to six carbon atoms and one to four heteroatoms selected from the group consisting of nitrogen, oxygen, and sulfur. For example, but not limiting, the term includes azetidine, pyrrolidine, piperidine, piperazine, morpholine, thiomorpholine, tetrahydropyran, tetrahydrofuran, hexahydropyrimidine, tetrahydropyrimidine, dihydroimidazole, and the like.

The term “optionally substituted C₃₋₆ heterocycloalkyl” refers to a C₃₋₆ heterocycloalkyl optionally substituted on the ring carbons with 1 to 4 substituents independently selected from the group consisting of optionally substituted C₁₋₄ alkyl, C₂₋₄ alkenyl, C₁₋₄ alkoxy, C₁₋₉ amide, C₁₋₇ amido, C₀₋₈ alkylamino, C₁₋₅ oxycarbonyl, cyano, C₃₋₈ cycloalkyl, C₃₋₈ cycloalkoxy, halo, hydroxy, nitro, oxo, and optionally substituted phenyl; and optionally substituted on any ring nitrogen with a substituent selected from the group consisting of optionally substituted C₁₋₄ alkyl, C₂₋₄ alkenyl, C₃₋₈ cycloalkyl, optionally substituted C₃₋₆ heterocycloalkyl, optionally substituted C₁₋₁₀ heteroaryl, and optionally substituted phenyl. A particular “optionally substituted C₃₋₆ heterocycloalkyl” is one optionally substituted on the ring carbons with 1 to 2 substituents independently selected from the group consisting of C₁₋₄ alkyl, C₁₋₄ alkoxy, and hydroxy; and optionally substituted on any ring nitrogen with a C₁₋₄ alkyl. A more particular “optionally substituted C₃₋₆ heterocycloalkyl” is one optionally substituted on any ring nitrogen with a C₁₋₄ alkyl.

The term “C₃₋₁₂ heterocycloalkyl” refers to a 4 to 16 membered monocyclic or bicyclic (including spiro used) saturated or partially (but not fully) unsaturated ring(s) having three to twelve carbon atoms and one to four heteroatoms selected from the group consisting of nitrogen, oxygen, and sulfur. For example, but not limiting, the term includes azetidine, pyrrolidine, piperidine, piperazine, morpholine, tetrahydropyran, tetrahydrofuran, hexahydropyrimidine, tetrahydropyrimidine, dihydroimidazole, oxa-3-azabicyclo[3.2.1]octane 8-oxa-3-azabicyclo[3.2.1]octane, 3,8-diazabicyclo[3.2.1]octane, 2-oxa-5-azabicyclo[2.2.1]heptane, N-10-oxa-4-azatricyclo[5.2.1.0^{2,6}]decane, dihydropyran, tetrahydropyran, and the like.

The term “optionally substituted C₃₋₁₂ heterocycloalkyl” refers to a C₃₋₁₂ heterocycloalkyl optionally substituted on the ring carbons with 1 to 5 substituents independently selected from the group consisting of optionally substituted C₁₋₄ alkyl, C₂₋₄ alkenyl, C₁₋₄ alkoxy, C₁₋₉ amide, C₁₋₇ amido, C₀₋₈ alkylamino, C₁₋₅ oxycarbonyl, cyano, C₃₋₈ cycloalkyl, C₃₋₈ cycloalkoxy, halo, hydroxy, nitro, oxo, and optionally substituted phenyl; and optionally substituted on any ring nitrogen with a substituent selected from the group consisting of optionally substituted C₁₋₄ alkyl, C₂₋₄ alkenyl, C₃₋₈ cycloalkyl, optionally substituted C₃₋₆ heterocycloalkyl, optionally substituted C₁₋₁₀ heteroaryl, and optionally substituted phenyl.

The term “C₁₋₁₀ heteroaryl” refers to a five to twelve membered monocyclic and polycyclic having unsaturated, conjugated ring(s) having aromatic character and having one to ten carbon atoms and one or more, typically one to four, heteroatoms selected from the group consisting of nitrogen, oxygen, and sulfur. For example, but not limiting, the term includes azepine, diazepine, furan, thiophene, imidazole, isothiazole, isoxazole, oxadiazole, oxazole, pyrazine, pyrazole, pyridazine, pyridine, pyrimidine, thiazole, thiadiazole, triazole, tetrazole, benzazepine, benzodiazepine, benzofuran, benzothiophene, benzimidazole, imidazopyridine, pyrazolopyridine, pyrrolopyridine, quinazoline, thienopyridine, indolizine, imidazopyridine, quinoline, isoquinoline, indole, isoindole, benzoxazole, benzoxadiazole, benzopyrazole, benzothiazole, and the like.

The term “optionally substituted C₁₋₁₀ heteroaryl” refers to a C₁₋₁₀ heteroaryl optionally having 1 to 5 substituents on carbon independently selected from the group consisting of C₁₋₇ amido, C₀₋₈ alkylamino, C₁₋₉ amide, C₂₋₅ carbamoyl, C₁₋₆ sulfonylamido, C₀₋₆ sulfonylamino, C₁₋₅ ureido, optionally substituted C₁₋₄ alkyl, optionally substituted C₁₋₄ alkoxy, cyano, halo, hydroxyl, oxo, nitro, C₁₋₅ oxycarbonyl, and C₁₋₈ sulfonyl and optionally having substituents on each nitrogen independently selected from the group consisting of optionally substituted C₁₋₄ alkyl, C₁₋₈sulfonyl, optionally substituted C₃₋₆ heterocycloalkyl, and optionally substituted phenyl. A particular “optionally substituted C₁₋₁₀ heteroaryl” is one optionally having 1 to 3 substituents on carbon independently selected from the group consisting of C₀₋₈ alkylamino, C₁₋₉ amide, C₂₋₅ carbamoyl, C₁₋₄ alkyl, C₁₋₄ alkoxy, cyano, halo, hydroxyl, oxo, trifluoromethyl, and trifluoromethoxy and optionally having on each nitrogen independently a C₁₋₄ alkyl.

The term “oxo” refers to an oxygen atom having a double bond to the carbon to which it is attached to form the carbonyl of a ketone or aldehyde. It is understood that as the term is used herein oxo refers to doubly bonded oxygen attached to the group which has the oxo substituent, as opposed to the oxo group being pendant as a formyl group. For example, an acetyl radical is contemplated as an oxo substituted alkyl group and a pyridone radical is contemplated as oxo substituted C₁₋₁₀ heteroaryl.

The term “C₁₋₁₀ heteroaryloxy” refers to a C₁₋₁₀ heteroaryl attached through an oxygen.

The term “optionally substituted C₁₋₁₀ heteroaryloxy” refers to a C₁₋₁₀ heteroaryl optionally having 1 to 5 substituents on carbon independently selected from the group consisting of C₁₋₄ alkyl, C₁₋₄ alkoxy, cyano, halo, hydroxyl, nitro, oxo, C₁₋₈ sulfonyl, and trifluoromethyl and optionally having substituents on each nitrogen independently selected from the group consisting of optionally substituted C₁₋₄ alkyl, C₁₋₈ sulfonyl, and optionally substituted phenyl.

The term “optionally substituted phenyl” refers to a phenyl group optionally having 1 to 5 substituents independently selected from the group consisting of C₂₋₄ alkenyl, C₁₋₄ alkyl, C₁₋₄ alkoxy, C₁₋₉ amide, C₀₋₈ alkylamino, C₁₋₅ oxycarbonyl, cyano, halo, hydrogen, hydroxyl, nitro, C₁₋₈ sulfonyl, and trifluoromethyl.

The term “C₁₋₆ sulfonylamido” refers to a —NHS(O)₂—R group wherein R is C₁₋₆ alkyl.

The term “C₀₋₆ sulfonylamino” refers to a —S(O)₂NH—R group wherein R is selected from the group consisting of hydrogen and C₁₋₆ alkyl.

The term “C₁₋₄ thioalkoxy” refers to a C₁₋₄ alkyl attached through a sulfur atom.

The term “C₂₋₅ carbamoyl” refers to an O- or N-linked carbamate having a terminal C₁₋₄ alkyl.

The term “C₁₋₅ ureido” refers to a urea optionally having an N—C₁₋₄ alkyl.

The term “pharmaceutically acceptable salt” refers to salts of pharmaceutically acceptable organic acids and bases or inorganic acids and bases. Such salts are well known in the art and includes those described in Journal of Pharmaceutical Science, 66, 2-19 (1977). Examples are the hydrochloride and mesylate salts.

It is understood that, where the terms defined herein mention a number of carbon atoms, that the mentioned number refers to the mentioned group and does not include any carbons that may be present in any optional substituent(s).

The skilled artisan will appreciate that certain of the compounds of the present invention exist as isomers. All mixtures of stereoisomers, in any ratio, and specific geometric isomers, enantiomers, and diastereomers of the compounds of the invention are contemplated to be within the scope of the present invention.

The skilled artisan will appreciate that certain of the compounds of the present invention exist as tautomers. All tautomeric forms the compounds of the invention are contemplated to be within the scope of the present invention.

The term “compounds of the invention” include the embodiment of formula I and the other embodiments and examples described herein.

a. One embodiment relates to compounds of formula I wherein Ar is C₄₋₁₄ aryl.

b. Another embodiment relates to compounds of formula I wherein Ar is phenyl.

c. Another embodiment relates to compounds of formula I and embodiments a and b wherein m is 1 or 2.

d. Another embodiment relates to compounds of formula I and embodiments a, b, and c wherein R₃ and R₄ are taken together along with the carbon to which they are attached to form an optionally substituted spiro ing selected from the group consisting of:

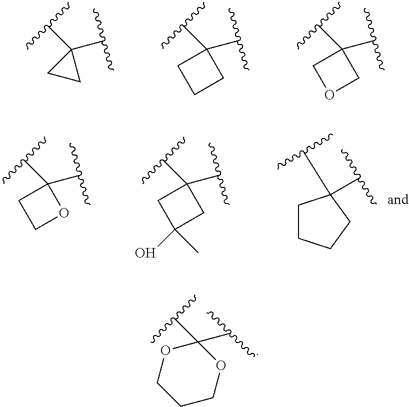

e. Another embodiment relates to compounds of formula I and embodiments a, b, and c wherein R₃ is C₁₋₆ alkyl.

f. Another embodiment relates to compounds of formula I and embodiments a, b, c, and e wherein R₄ is C₁₋₆ alkyl.

g.1. Another embodiment relates to compounds of formula I and embodiments a, b, and c wherein R₃ and R₄ are methyl.

g.2. Another embodiment relates to compounds of formula I and embodiments a, b, and c wherein R₃ and R₄ are taken together along with the carbon to which they are attached to form the spiro ring shown below:

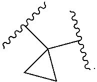

h. Another embodiment relates to compounds of formula I and embodiments a, b, c, d, e, f, g.1, and g.2 wherein G₁ is O and G₂ is CR₅R₆.

i. Another embodiment relates to compounds of formula I and embodiments a, b, c, d, e, f, g.1, and g.2 wherein G₂ is O and G₁ is CR₅R₆.

j. Another embodiment relates to compounds of formula I and embodiments a, b, c, d, e, f, g.1, g.2, h, and i wherein R₅ and R₆ are hydrogen.

k. Another embodiment relates to compounds of formula I and embodiments a, b, c, d, e, f, g.1, g.2, h, i, and j wherein R₂, is, each time taken, independently selected from the group consisting of cyano, optionally substituted C₁₋₆ alkyl, C₁₋₈ sulfonyl, optionally substituted C₂₋₄ alkenyl, optionally substituted C₂₋₄ alkynyl, optionally substituted C₁₋₄ alkoxy, C₀₋₈ alkylamino, optionally substituted C₄₋₁₄ aryl, optionally substituted C₄₋₁₄ aryloxy, C₁₋₅ oxycarbonyl, C₁₋₅ carbonyloxy, cyano, optionally substituted C₃₋₆ heterocycloalkyl, optionally substituted C₁₋₁₀ heteroaryl, hydroxy, nitro, —NHC(O)NR₇R₈, —NHC(O)OR₉, —NH(SO₂)NHR₇, —NHC(O)NHNR₇R₈, —NHC(S)NR₇R₈, —NHC(═NR₁₀)NR₇R₈, —NHC(SR₁₁)NR₇R₈, and —NHC(═NR₁₀)OR₁₂.

1.1 Another embodiment relates to compounds of formula I and embodiments a, b, c, d, e, f, g.1, g.2, h, i, j, and k wherein R₁ is optionally substituted C₃₋₁₂ heterocycloalkyl.

1.2 Another embodiment relates to compounds of formula I and embodiments a, b, c, d, e, f, g.1, g.2, h, i, j, and k wherein R₁ is C₃₋₁₂ heterocycloalkyl.

m. Another embodiment relates to compounds of formula I and embodiments a, b, c, d, e, f, g.1, g.2, h, i, j, and k wherein R₁ is N-morpholinyl.

n. Another embodiment relates to compounds of formula I and embodiments a, b, c, d, e, f, g.1, g.2, h, i, j, and k wherein R₁ is N-8-oxa-3-azabicyclo[3.2.1]octanyl.

o. Another embodiment relates to compound of formula I and embodiments a, b, c, e, f,

g.1, g.2, h, i, j, k, 1.1, 1.2, m, and n wherein m is 1 and R₂, is —NHC(O)NR₇R₈.

p. Another embodiment relates to each embodiment of o, above, wherein R₇ is hydrogen and R₈ is C₁₋₄ alkyl.

The compounds of the invention can be prepared by a variety of procedures, some of which are described below. All substituents, unless otherwise indicated, are as previously defined. The products of each step can be recovered by conventional methods including extraction, evaporation, precipitation, chromatography, filtration, trituration, crystallization, and the like. The procedures may require protection of certain groups, for example hydroxy, amino, or carboxy groups to minimize unwanted reactions. The selection, use, and removal of protecting groups is well known and appreciated as standard practice, for example T. W. Greene and P. G. M. Wuts in Protective Groups in Organic Chemistry (John Wiley and Sons, 1991).

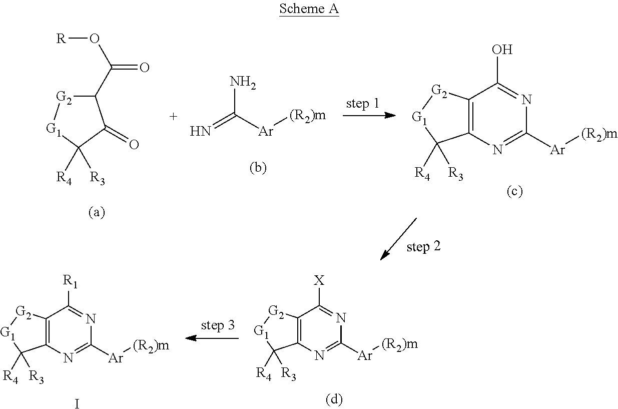

Scheme A, step 1, depicts the reaction of an appropriate compound of formula (a) with an appropriate compound of formula (b) to give a compound of formula (c). An appropriate compound of formula (a) is one in which R is C₁₋₄ alkyl and G₁, G₂, R₃, and R₄, are as desired in the final compound of formula I or gives rise to R₃ and R₄ as desired in the final compound of formula I. The preparation of compounds of formula (a) is readily appreciated in the art. An appropriate compound of formula (b) is one in which Ar, R₂, and m, are as desired in the final compound of formula I or gives rise to R₂ and m as desired in the final compound of formula I. Appropriate compounds of formula (b) are readily available and may be used in step 1 in the form of their salts, including their hydrochloride salt. Such condensation reactions are well understood and appreciated in the art.

For example, is carried out in a polar solvent such as water, methanol, ethanol, isopropanol at temperatures of from around 10° C. to 150° C., and typically require 2 to 12 hours. The reaction is generally carried out in the presence of base, particularly when a salt is used. The reaction may be carried out using microwave radiation. Suitable bases include alkali metal hydroxides, such as sodium hydroxide, and alkali metal alkoxides, such as sodium alkoxides, and the like.

Scheme A, step 2, depicts the reaction of a compound of formula (c) with an appropriate activating reagent to give a compound of formula (d) in which X is an activating group. A variety of activating groups are readily formed and are suitable. For example, X can be triflate and halogen, particularly chloro, bromo, and iodo.

For example, the reaction typically uses an excess of the selected suitable halogen converting agent is carried out in a solvent, such as dichloromethane, THF, acetonitrile, and the like. Suitable halogen converting reagents capable of converting a hydroxyl to halogen, such as, phosphorous oxychloride, phosphorous trichloride, phosphorous pentachloride, phosphorous pentabromide, phosphorous oxybromide, phosphorous tribromide, thionyl chloride, thionyl bromide, bromine/triphenylphosphine, and the like are well known in the art. In some cases the halogen converting reagent can be used as a solvent. The reaction may be carried out using microwave radiation. The reaction is typically carried out at temperatures of from 0° C. to reflux temperature of the selected solvent and typically require 1 to 15 hours.

Scheme A, step 3, depicts the reaction of a compound of formula (d) to give a compound of formula I.

For example, a compound of formula (d) is reacted with an appropriate compound H—R₁ to give a compound of formula I. An appropriate compound H—R₁ is in which R₁ is a nitrogen containing optionally substituted C₃₋₁₂ heterocycloalkyl I and the H is attached to a nitrogen. Such a reaction is generally carried out in a solvent, such as dichloromethane, THF, dimethylformamide, dimethylacetamide, and the like. The reaction is carried out with the use of a suitable base. Bases such as triethylamine are typically used. The reaction may be carried out using microwave radiation. The reaction is typically carried out at temperatures of from 50° C. to 150° C. The reaction typically requires 1 to 72 hours.

Also, for example, a compound of formula (d) is reacted with an appropriate compound (HO)₂B—R₁ to give a compound of formula I. An appropriate compound (HO)₂B—R₁ is one in which R₁ is as desired in the final compound of formula I or gives rise to R₁ as desired in the final compound of formula I. Such reactions are carried out using transition metal catalysts, for example, the Suzuki coupling which is well known in the art.

It is also understood that there are variations of the Scheme A above, particularly for the introduction of R₂ as desired in the final compound of formula I. It is also understood that some compounds of formula I may be elaborated to other compounds of formula I, in an additional steps not shown. For example, a compound of formula I in which R₂ is halogen, generally bromo, can undergo a variety of reactions to give compound in which R₂ is other than halogen. Compounds of formula I may be elaborated in a variety of other ways, in an additional step not shown. Such reactions include hydrolysis, oxidation, reduction, alkylation, amidations, sulfonations, alkynations, alkyenations, and the like. Also, in an optional step, not shown, the compounds of formula I can be converted to pharmaceutically acceptable salts by methods well known and appreciated in the art.

The present invention is further illustrated by the following examples and preparations. the examples and preparations do not limit the scope of the invention in any way. The terms and abbreviations used in the examples have their usual meaning unless otherwise indicated. For example, DMF is dimethyl formamide, DCM is dichloromethane, ACN is acetonitrile, etc. Typically HPLC was carried out by reverse phase on a column (Gemini 5₁μ C18 110A, AXIA, 30×75 mm, 5 micron) and eluted with gradients of ACN (containing 0.035% TFA) and water (containing 0.05% TFA). Product was generally recovered by evaporation in vacuo.

### Example 1

### 2-(4-bromophenyl)-7,7-dimethyl-4-morpholino-6,7-dihydrofuro[3,2-d]pyrimidine

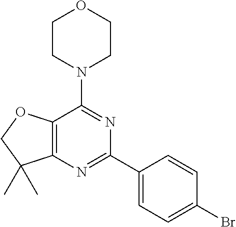

In a microwave vial, ethyl 4,4-dimethyl-3-oxotetrahydrofuran-2-carboxylate. 4-bromobenzimidamide hydrochloride (2.150 g, 9.13 mmol) and sodium methoxide in methanol, 30% (8.56 ml, 45.6 mmol) were combined in anhydrous methanol (8 ml) and the reaction mixture was sealed and heated in a 120° C. oil bath for 18 hours. The reaction mixture was then cooled and diluted with ethyl acetate (150 ml) and washed with saturated sodium chloride solution. The aqueous layer was extracted one more time with ethyl acetate, the organic layers combined, dried over sodium sulfate and concentrated to near dryness to give a solid. The solid was triturated with a mixture of ethyl acetate/hexane was added (10 ml, about 1:1). The solid was filtered, washed with hexane to give 2-(4-bromophenyl)-7,7-dimethyl-6,7-dihydrofuro[3,2-d]pyrimidin-4-ol as a white solid (0.375 g, 12.8% yield). MS [M+H] found 321-323. ¹H NMR (400 MHz, MeOD) δ 1.39 (s, 6H), 4.40 (s, 2H), 7.64-7.71 (m, 2H), 7.84-7.93 (m, 2H).

In a microwave vial, 2-(4-bromophenyl)-7,7-dimethyl-6,7-dihydrofuro[3,2-d]pyrimidin-4-ol (0.375 g, 1.168 mmol) and phosphorus oxychloride (3 ml, 32.8 mmol) were combined and the reaction mixture was heated in the microwave at 100° C. for 30 minutes. The reaction mixture was concencentrated in vacuo to give a residue and the residue was dissolved in ethyl acetate, poured into a beaker of ice and saturated sodium carbonate solution, the two layers separated. The aqueous layer was extracted one more time with ethyl acetate, the combined organic layers were washed with brine, dried over sodium sulfate and concentrated to give 2-(4-bromophenyl)-4-chloro-7,7-dimethyl-6,7-dihydrofuro[3,2-d]pyrimidine as a tan solid: 0.350 g, 88.0% yield. MS [M+H] found 339 and 341.

A mixture of 2-(4-bromophenyl)-4-chloro-7,7-dimethyl-6,7-dihydrofuro[3,2-d]pyrimidine (0.170 g, 0.501 mmol), morpholine (0.438 ml, 5.00 mmol) and triethylamine (0.908 ml, 6.51 mmol) in anhydrous dichloromethane (2 ml) was heated in the microwave at 110° C. for 2 hours. The reaction mixture was concentrated and purified on silica gel column eluting with a gradient of 5-20% ethyl acetate in hexane to give of the title compound: (0.112 g, 57.3% yield). MS [M+H] found 390 and 392. ¹H NMR (400 MHz, chloroform-d) δ 1.39 (s, 6H), 3.78-3.85 (m, 4H), 3.86-3.94 (m, 4H), 4.29 (s, 2H), 7.50-7.57 (m, J=8.59 Hz, 2H), 8.19-8.28 (m, J=8.59 Hz, 2H).

### Example 2

### 1-(4-(7,7-dimethyl-4-morpholino-6,7-dihydrofuro[3,2-d]pyrimidin-2-yl)phenyl)-3-methylurea

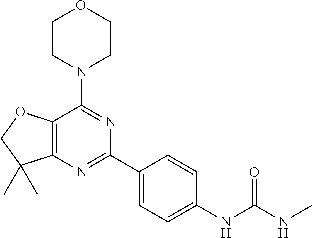

In a microwave vial 2-(4-bromophenyl)-7,7-dimethyl-4-morpholino-6,7-dihydrofuro[3,2-d]pyrimidine (0.060 g, 0.154 mmol), 1-methylurea (0.171 g, 2.306 mmol), copper(I) iodide (9.96 mg, 0.052 mmol), Xantphos (0.018 g, 0.031 mmol), tris(dibenzylideneacetone)dipalladium (0) (0.014 g, 0.015 mmol), sodium tert-butoxide (0.222 g, 2.306 mmol), and degassed dioxane (2.2 ml) were combined. The reaction vial was flushed with nitrogen for 7 minutes then heated in the microwave at 110° C. for 1 hour. The reaction mixture cooled and purified by preparative HPLC eluting with a gradient 30-70% ACN (containing 0.035% TFA) in water (containing 0.05% TFA) to give the title compound as its TFA salt: 0.0415 g, 70.4% yield. MS [M+H] found 384. ¹H NMR (400 MHz, MeOD) δ 1.54 (s, 6H), 2.79 (s, 3H), 3.75-3.90 (m, 4H), 4.12-4.25 (m, 4H), 4.49 (s, 2H), 7.55-7.64 (m, J=8.84 Hz, 2H), 7.94-8.06 (m, J=8.84 Hz, 2H).

### Example 3

### 4-(8-oxa-3-azabicyclo[3.2.1]octan-3-yl)-2-(4-bromophenyl)-7,7-dimethyl-6,7-dihydrofuro[3,2-d]pyrimidine

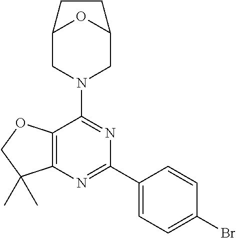

The title compound was prepared by a method similar to Example 1 using 8-oxa-3-azabicyclo[3.2.1]octane hydrochloride salt.

### Example 4

### 1-(4-(4-(8-oxa-3-azabicyclo[3.2.1]octan-3-yl)-7,7-dimethyl-6,7-dihydrofuro[3,2-d]pyrimidin-2-yl)phenyl)-3-methylurea

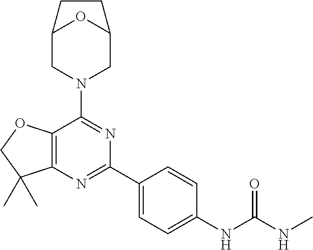

The title compound was prepared by a method similar to Example 2 and isolated as its

TFA salt: MS [M+H] calculated for C₂₂H₂₇N₅O₃, 410; found 410. ¹H NMR (400 MHz, MeOD) δ 1.53 (s, 6H), 1.85 (d, J=7.33 Hz, 2H), 1.96-2.08 (m, 2H), 2.80 (s, 3H), 3.48 (d, J=12.88 Hz, 2H), 4.50 (s, 4H), 4.74 (d, J=13.39 Hz, 2H), 7.57-7.66 (m, 2H), 7.97-8.05 (m, 2H).

### Example 5

### 4-(8-oxa-3-azabicyclo[3.2.1]octan-3-yl)-2-(4-bromophenyl)-7,7-dimethyl-5,7-dihydrofuro[3,4-d]pyrimidine

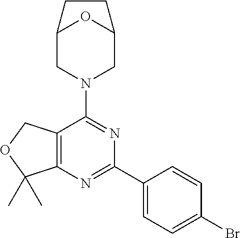

Sodium hydride (60%, 1.220 g, 30.5 mmol) and ether (19 mL) were placed in a round-bottom flask. Methyl 2-hydroxy-2-methylpropanoate (3.603 g, 30.5 mmol) was slowly dripped into the suspension at 0° C. The resulting solution was stirred at ambient temperature for 10 minutes, followed by removing ether under reduced pressure with an evaporator. DMSO (12 mL) was added to the dried residue immediately followed by adding methyl acrylate (3.02 mL, 33.6 mmol) all at once. The resulting solution was stirred at ambient temperature for 15 minutes and then 5 N HCl aqueous solution (62 mL) was added. The reaction mixture was extracted with ether (62 mL×2). The ether layers were washed with distilled water and dried over anhydrous sodium sulfate, evaporated to give a yellow oil. The oil was purified by distillation to give methyl 5,5-dimethyl-4-oxotetrahydrofuran-3-carboxylate: b.p.=61°-63° C. at 5 mm Hg (1.703 g, 9.89 mmol, 32.4% yield) as a colorless oil.

A mixture of methyl 5,5-dimethyl-4-oxotetrahydrofuran-3-carboxylate (1 g, 5.81 mmol), 4-bromo-benzamidine hydrochloride (2.74 g, 11.62 mmol) and aqueous NaOH (1N) (15 mL) was heated in microwave at 100° C. for 12 hours. 4-Bromo-benzamidine hydrochloride (2.74 g, 11.62 mmol) was added and the reaction was heated in microwave at 100° C. for 12 hours. The mixture was cooled to 0° C., adjusted with concentrated HCl to pH 7 and extracted with EtOAc. Concentration of the combined organic layers gave a solid, which was triturated in EtOAc and filtered. The filtrate was concentrated, triturated with DCM and filtered. The filtrate was and added to a silica gel column and was eluted with 30% hexanes and 70% EtOAc to give 2-(4-bromophenyl)-7,7-dimethyl-5,7-dihydrofuro[3,4-d]pyrimidin-4-ol (175 mg, 0.545 mmol, 9.38% yield) was obtained as a white solid. MS [M+H] found 321.

2-(4-Bromophenyl)-7,7-dimethyl-5,7-dihydrofuro[3,4-d]pyrimidin-4-ol (175 mg, 0.545 mmol) and phosphoryl trichloride (1.4 mL, 15.29 mmol) were combined and the reaction mixture was heated in the microwave at 100° C. for 30 minutes. The mixture was cooled and then concencentrated in vacuo to give a residue. The residue was dissolved in ethyl acetate, poured into ice and saturated sodium carbonate solution. The two layers were separated. The aqueous layer was extracted one more time with ethyl acetate, then the combined organic layers were washed with brine, dried over sodium sulfate, and concentrated to give 2-(4-bromophenyl)-4-chloro-7,7-dimethyl-5,7-dihydrofuro[3,4-d]pyrimidine (193.2 mg, 0.569 mmol, quantitative yield) as a white solid. MS [M+H] found 339.

A mixture of 2-(4-bromophenyl)-4-chloro-7,7-dimethyl-5,7-dihydrofuro[3,4-d]pyrimidine (97 mg, 0.286 mmol), 8-oxa-3-azabicyclo[3,2,1]octane hydrochloride (86 mg, 0.574 mmol) and triethylamine (0.2 mL, 1.435 mmol) in anhydrous dichloromethane (2 mL) was heated in microwave at 120° C. for 1 hour and then at 110° C. for 12 h. 8-Oxa-3-azabicyclo[3.2.1]octane hydrochloride (86 mg, 0.574 mmol) and triethylamine (0.2 mL, 1.435 mmol) were added and the reaction was heated in the microwave at 120° C. for an additional 1 hour. The reaction mixture was cooled, concentrated in vacuo, and purified on a silica gel column with a gradient of 5-20% ethyl acetate in hexanes to give the title compound (75 mg, 0.180 mmol, 62.8% yield) as a white solid. MS [M+H] found 416.

### Example 6

### 1-(4-(4-(8-oxa-3-azabicyclo[3.2.1]octan-3-yl)-7,7-dimethyl-5,7-dihydrofuro[3,4-d]pyrimidin-2-yl)phenyl)-3-methylurea

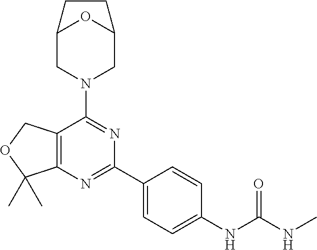

To a microwave tube were added 4-(8-oxa-3-azabicyclo[3.2.1]octan-3-yl)-2-(4-bromophenyl)-7,7-dimethyl-5,7-dihydrofuro[3,4-d]pyrimidine (75 mg, 0.180 mmol), 1-methylurea (200 mg, 2.70 mmol), copper(I) iodide (11.67 mg, 0.061 mmol), xantphos (20.85 mg, 0.036 mmol), tris(dibenzylidineacetone)dipalladium(0) (16.50 mg, 0.018 mmol), sodium tert-butoxide (260 mg, 2.70 mmol) and degassed dioxane (2 mL). The reaction tube was flushed with nitrogen for 7 m then heated in the microwave at 110° C. for 20 minutes. Methanol was added to the reaction mixture. The mixture was filtered and the filtrate was purified by HPLC eluting with a gradient 20-25% ACN (containing 0.035% TFA) in water (containing 0.05% TFA) to give the title compound (27 mg, 0.066 mmol, 36.6% yield) as its TFA salt. ¹H NMR (400 MHz, methanol-d4) δ ppm 1.65 (s, 6H) 1.90-1.97 (m, 2H) 2.05-2.11 (m, 2H) 2.88 (s, 3H) 3.48-3.57 (m, 2H) 4.24 (dd, J=9.47, 3.92 Hz, 2H) 4.58 (d, J=2.27 Hz, 2H) 5.32 (s, 2H) 7.65 (d, J=8.84 Hz, 2H) 8.22 (d, J=8.84 Hz, 2H). MS [M+H] found 410.

### Example 7

### 2-(4-bromophenyl)-7,7-dimethyl-4-morpholino-5,7-dihydrofuro[3,4-d]pyrimidine

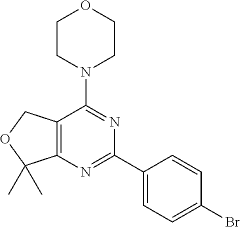

The title compound was prepared by a method similar to Example 1 using morpholine.

### Example 8

### 1-(4-(7,7-dimethyl-4-morpholino-5,7-dihydrofuro[3,4-d]pyrimidin-2-yl)phenyl)-3-methylurea

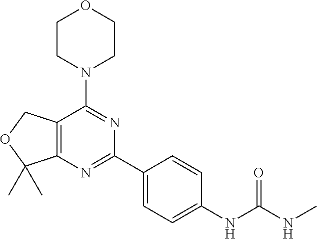

The title compound was prepared by a method similar to Example 6 and isolated as its TFA salt. ¹H NMR (400 MHz, methanol-d4) δ ppm 1.60 (s, 6H) 2.87 (s, 3H) 3.88 (s, 8H) 5.28 (s, 2H) 7.59 (d, J=8.84 Hz, 2H) 8.28 (d, J=8.84 Hz, 2H). MS [M+H] found 384.

### Example 9

### 1-(4-(4-(8-oxa-3-azabicyclo[3.2.1]octan-3-yl)-7,7-dimethyl-5,7-dihydrofuro[3,4-d]pyrimidin-2-yl)phenyl)-3-ethylurea

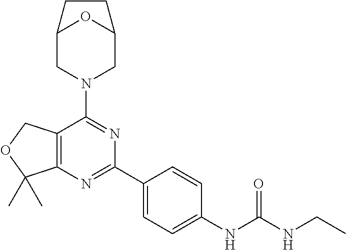

The title compound was prepared by a method similar to Example 6 except 1-ethylurea was used. ¹H NMR (400 MHz, DMSO-d6) δ ppm 1.06 (t, J=8 Hz, 3H) 1.40 (s, 6H) 1.76-1.85 (m, 4H) 3.12 (m, 2H) 3.22 (m, 2H) 3.93 (m, 2H) 4.42 (m, 2H) 5.15 (s, 2H) 6.16 (br s, 1H) 7.49 (d, J=8 Hz, 2H) 8.19 (J=8 Hz, 2H) 8.67 (s, 1H). MS [M+H] found 424.

### Example 10

### 14447 ,7-dimethyl-4-morpholino-5,7-dihydrofuro[3,4-d]pyrimidin-2-yl)phenyl)-3-ethylurea

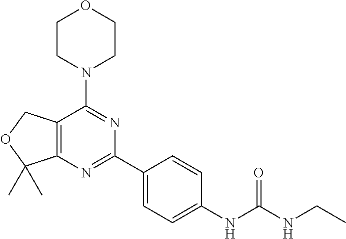

The title compound was prepared by a method similar to Example 6 except 1-ethylurea was used. ¹H NMR (400 MHz, methanol-d4) δ ppm 1.25 (t, J=8 Hz, 3H) 1.65 (s, 6H) 3.32 (q, J=8 Hz, 2H) 3.89-3.94 (m, 8H) 5.31 (s, 2H) 7.64 (d, J=8 Hz, 2H) 8.22 (d, J=8 Hz, 2H). MS [M+H] found 398.

### Example 11

### 1-cyclopropyl-3-(4-(7,7-dimethyl-4-morpholino-5,7-dihydrofuro[3,4-d]pyrimidin-2-yl)phenyl)urea

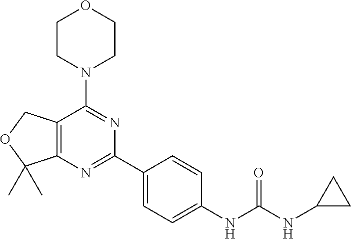

The title compound was prepared by a method similar to Example 6 except 1-cyclopropylurea was used. ¹H NMR (400 MHz, methanol-d4) δ ppm 0.60 (m, 2H) 0.83 (m, 2H) 1.64 (s, 6H) 2.69 (m, 1H) 3.90 (m, 8H) 5.31 (s, 2H) 7.65 (d, J=8 Hz, 2H) 8.25 (d, J=8 Hz, 2H). MS [M+H] found 410.

### Example 12

### 1-(4-(4-(8-oxa-3-azabicyclo[3.2.1]octan-3-yl)-7-hydroxy-7-methyl-5,7-dihydrofuro[3,4-d]pyrimidin-2-yl)phenyl)-3-methylurea

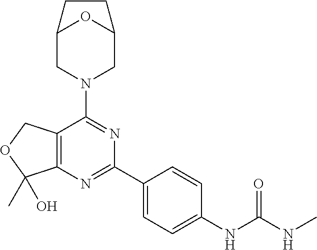

To a slurry of 4-(8-oxa-3-azabicyclo[3.2.1]octan-3-yl)-2-chlorofuro[3,4-d]pyrimidin-7(5H)-one (308 mg, 1.09 mmol) in THF (10 mL) at 0° C. was slowly added methylmagnesiumbromide (364 μL, 1.09 mmol, 3 M in ether). The mixture was stirred at 0° C. for 50 min and then more methylmagnesiumbromide (364 μL) was added. After another 5 min at 0° C., it was quenched with NH₄Cl (sat.). THF was reduced by a stream of air and the resulting mixture was extracted with CH₂Cl₂, dried over Na₂SO₄. Solvent was evaporated to give crude 4-(8-oxa-3-azabicyclo[3.2.1]octan-3-yl)-2-chloro-7-methyl-5,7-dihydrofuro[3,4-d]pyrimidin-7-ol (149 mg, 46%). MS [M+H] found 298.

To 4-(8-oxa-3-azabicyclo[3.2.1]octan-3-yl)-2-chloro-7-methyl-5,7-dihydrofuro[3,4-d]pyrimidin-7-ol (85 mg, 0.285 mmol) was added HCl in MeOH (1.25 M, 4 mL). The mixture was stirred at room temperature for 30 min and the solvent was reduced by a stream of air and subjected to vacuum for 10 min to give a light yellow solid. To the solid was added 1-Methyl-3-[4-(4,4,5,5-Tetramethyl-1,3,2-dioxaborolane-2-yl)phenyl]-urea (118 mg, 0.428 mmol), PdCl₂(dppf)-CH₂Cl₂ (cat.), 1,4-Dioxane (2 ml), and NaHCO₃ aq. (1 ml). The mixture was heated in a microwave at 120° C. for 50 min. The mixture was passed through filter and washed with MeOH. The filtrate was purified by preparative HPLC (gradient 15-25% ACN (containing 0.035% TFA) in water (containing 0.05% TFA) to give the title compound as a yellow film (3.2 mg). MS [M+H] found 412.

### Example 13

### 1-(4-(4′-(8-oxa-3-azabicyclo[3.2.1]octan-3-yl)-5′H-spiro[cyclopropane-1,7′-furo[3,4-d]pyrimidine]-2′-yl)phenyl)-3-methylurea

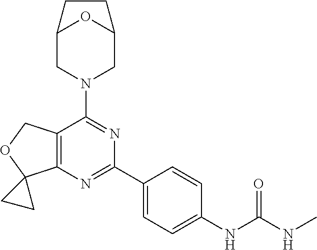

2,6-Dihydroxypyrimidine-4-carboxylic acid (100 g, 73.4 mmol) and paraformaldehyde (84 g, 293 mmol) were combined with concentrated HCl (1.5 L) and heated to reflux at 85-95° C. for 18 hours. The reaction was then cooled and HCl was evaporated under reduced pressure to obtain a solid. The solid was washed with petroleum ether to give crude 2,4-dihydroxyfuro[3,4-d]pyrimidin-7(5H)-one (90 g, 83% yield) which was used next tep without further purification.

A mixture of 2,4-dihydroxyfuro[3,4-d]pyrimidin-7(5H)-one (100 g, 595 mmol), phosphoryl trichloride (800 mL) and N,N-diethylaniline (150 mL) was heated at 110° C. for 18 hours. The reaction was cooled to ambient temperature, the solvent was removed in vacuo to give a residue. The residue was purified on a silica gel column eluted with petrolium ether:EtOAc=5:1 to give 2,4-dichlorofuro[3,4-d]pyrimidin-7(5H)-one (70 g, 57% yield) as an off-white solid.

A solution of 2,4-dichlorofuro[3,4-d]pyrimidin-7(5H)-one (34 g, 165 mmol) in DCM (770 mL) was added 8-oxa-3-azabicyclo[3.2.1]octane hydrochloride (24 g, 165 mmol) was cooled to 0° C. and then triethylamine (96 mL, 660 mmol) was added dropwise. The mixture was then concentrated in vacuo to give a residue which was purified on a silica gel column eluted with DCM:EtOAc=1:2 to give 4-(8-oxa-3-azabicyclo[3.2.1]octan-3-yl)-2-chlorofuro[3,4-d]pyrimidin-7(5H)-one (28 g, 82% yield) as a yellow solid. MS [M+H] found 282.

Under a nitrogen at 0° titanium chloride triisopropoxide (1 M in hexanes) (75 ml, 75 mmol) was added to a solution of 4-(8-oxa-3-azabicyclo[3.2.1]octan-3-yl)-2-chlorofuro[3,4-d]pyrimidin-7(5H)-one (19 g, 68 mmol) in THF (100 mL) followed by dropwise addition of ethylmagnesium bromide (1 M in THF) (150 ml, 150 mmol). The reaction was warmed up to ambient temperature and stirred overnight and then quenched by addition of a saturated NH₄Cl solution (200 mL). The water layer was extracted with ethyl acetate, and the combined organic layers were dried over Na₂SO₄, filtered, and evaporated in vacuo to give a residue. The residue was purified on a silica gel column eluted with petrolium ether:EtOAc=3:1 to give 1-(6-(8-oxa-3-azabicyclo[3.2.1]octan-3-yl)-2-chloro-5-(hydroxymethyl)pyrimidin-4-yl)cyclopropanol (5 g, 24% yield) as a yellow solid. MS [M+H] found 312.

To a solution of 1-(6-(8-oxa-3-azabicyclo[3.2.1]octan-3-yl)-2-chloro-5-(hydroxymethyl)pyrimidin-4-yl)cyclopropanol (3 g, 9.6 mmol) and PPh₃(2.5 g, 14.4 mmol) in dry THF (200 mL) was added DEAD (3.77 g, 14.4 mmol) dropwise at 0° C. The mixture was stirred at ambient temperature for 3 hours. The solvent was removed in vacuum to give a residue which was purified on silica gel column eluted with petroleum ether:EtOAc=4:1 to give a residue which was further purified by Prep HPLC (Column: Fuji C18 (300×25); Wavelength 220 nm; Mobile phase: A MeCN (0.1% TFA); B water (0.1% TFA); Flow rate: 25 mL/min; Injection volume: 2 mL; Run time: 20 min; Equilibration: 3 min) to give 4′-(8-oxa-3-azabicyclo[3.2.1]octan-3-yl)-2′-chloro-5′H-spiro[cyclopropane-1,7′-furo[3,4-d]pyrimidine] (330 mg, 12% yield). MS [M+H] found 294.

A mixture of 4′-(8-oxa-3-azabicyclo[3.2.1]octan-3-yl)-2′-chloro-5′H-spiro[cyclopropane-1,7′-furo[3,4-d]pyrimidine] (10.2 mg, 0.035 mmol), 1-Methyl-3-[4-(4,4,5,5-Tetramethyl-1,3,2-dioxaborolane-2-yl)phenyl]-urea (19.18 mg, 0.069 mmol), sat. NaHCO₃ aq. (0.25 ml, 0.035 mmol) and PdCl₂(dppf)-CH₂Cl₂ (2.269 mg, 2.78 μmol) in 1,4-Dioxane (0.5 ml) was heated by microwave at 110° C. for 30 min. The mixture was passed throughout filter and washed with MeOH. The filtrate was purified by preparative HPLC (gradient 20-45% ACN (containing 0.035% TFA) in water (containing 0.05% TFA). The fractions containing the desired compound were combined and concentrated in vacuo to give a solid. This solid was partitioned between EtOAc and NaHCO₃ aq. The phases were separated and the aqueous phase was extracted with EtOAc. The combined organic phases were washed with saturated aqueous NaCl, dried and concentrated in vacuo to give a beige solid, which was triturated with hexane/ethyl acetate (1:1), collected by filtration, rinsed with hexane/ethyl acetate (1:1) and dried to give the title compound (10.1 mg, 0.025 mmol, 71.4% yield) as a beige solid. ¹H NMR (400 MHz, DMSO-d6) δ ppm 1.09 (m, 2H) 1.18 (m, 2H) 1.78 (m, 4H) 2.65 (s, 3H) 3.25 (m, 2H) 3.95 (m, 2H) 4.43 (m, 2H) 5.32 (s, 2H) 6.12 (br s, 1H) 7.47 (d, J=8 Hz, 2H) 8.14 (d, J=8 Hz, 2H) 8.81 (s, 1H). MS [M+H] found 408.

### Example 14

### 1-ethyl-3-(4-(4′-morpholino-5′H-spiro[cyclopropane-1,7′-furo[3,4-d]pyrimidine]-2′-yl)phenyl)urea

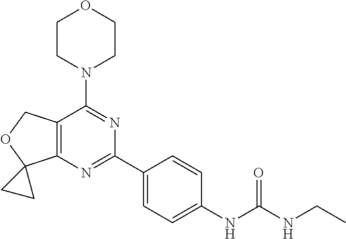

To a solution of 2,4-dichlorofuro[3,4-d]pyrimidin-7(5H)-one (34 g, 165 mmol) in DCM (770 mL) was added Morpholine (14.4 g, 165 mmol) and cooled to 0° C. Triethylamine (96 mL, 660 mmol) was added dropwise. The mixture was then concentrated in vacuo to give a residue which was purified on a silica gel column eluted with DCM:EtOAc=1:2 to give compound 2-chloro-4-morpholinofuro[3,4-d]pyrimidin-7(5H)-one (28 g, 67% yield) as a yellow solid. MS [M+H] found 256.

Titanium chloride triisopropoxide (1 M in hexanes) (81.7 ml, 81.7 mmol) was added to a solution of 2-chloro-4-morpholinofuro[3,4-d]pyrimidin-7(5H)-one (19 g, 74.3 mmol) in THF (100 mL) at 0° C. followed by dropwise addition of ethylmagnesium bromide (1 M in THF) (163.5 ml, 163.5 mmol). The reaction was warmed up to ambient temperature and stirred overnight, then quenched by addition of a saturated NH₄Cl solution (200 mL). The water layer was extracted with ethyl acetate, and the combined organic layers were dried over Na₂SO₄, filtered, and evaporated in vacuo to give a residue which was purified on a silica gel column eluted with petroleum ether:EtOAc=3:1 to give 1-(2-chloro-5-(hydroxymethyl)-6-morpholinopyrimidin-4-yl)cyclopropanol (8 g, 37.7% yield). MS [M+H] found 286.

DIAD (4.31 g, 21.35 mmol) was added dropwise to a solution of PPh₃(5.6 g, 21.35 mmol) in dry THF (200 mL) at 0° C. and stirred at room temperature for 1 hour, then cooled to 0° C. and 1-(2-chloro-5-(hydroxymethyl)-6-morpholinopyrimidin-4-yl)cyclopropanol (5 g, 17.5 mmol, 1 eq) in THF (60 ml) was added dropwise. The mixture was stirred warmed to room temperature and stirred for 14 hours before the solvent was removed in vacuum to give a residue which was purified on silica gel column eluted with petrolium ether:EtOAc=4:1 to give a residue which was further purified by Prep HPLC (Column: Fuji C18 (300×25); Wavelength 220 nm; Mobile phase: A MeCN (0.1% TFA); B water (0.1% TFA); Flow rate: 25 mL/min; Injection volume: 2 mL; Run time: 20 min; Equilibration: 3 min) to give 2′-chloro-4′-morpholino-5′H-spiro[cyclopropane-1,7′-furo[3,4-d]pyrimidine] (1.67 g, 24.8% yield). MS [M+H] found 268.

2′-Chloro-4′-morpholino-5′H-spiro[cyclopropane-1,7′-furo[3,4-d]pyrimidine] (200 mg, 0.747 mmol), 1-ethyl-3-(4-(4,4,5,5-tetramethyl-1,3,2-dioxaborolan-2-yl)phenyl)urea (325 mg, 1.12 mmol), Pd(dffp)C₁₂-DCM (cat.), 1,4-dioxane (6 mL), and NaHCO₃ (sat., 3 mL) were combined and heated in a microwave at 110° C. for 30 minutes, then diluted with MeOH and filtered through a microfilter. The filtrate was purified by preparative HPLC (gradient 30-30% ACN (containing 0.035% TFA) in water (containing 0.05% TFA) to give the title compound (24.1 mg, 0.061 mmol, 8% yield) as a white solid. ¹H NMR (400 MHz, Methanol-d4) δ ppm 1.17 (t, J=8 Hz, 3H) 8 (m, 4H) 3.26 (q, J=8 Hz, 2H) 3.85 (m, 8H) 5.37 (s, 2H) 7.55 (d, J=8 Hz, 2H) 8.08 (d, J=8 Hz, 2H). MS [M+H] found 396.

### Example 15

### 1-cyclopropyl-3-(4-(4′-morpholino-5′H-spiro[cyclopropane-1,7′-furo[3,4-d]pyrimidine]-2′-yl)phenyl)urea

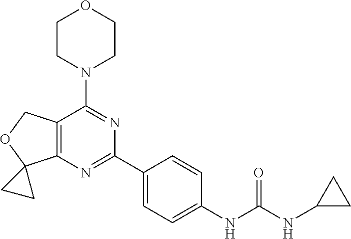

The title compound was prepared by a method similar to Example 14 except1-cyclopropyl-3-(4-(4,4,5,5-tetramethyl-1,3,2-dioxaborolan-2-yl)phenyl)urea was used. ¹H NMR (400 MHz, DMSO-d6) δ ppm 0.41 (m, 2H) 0.63 (m, 2H) 1.07 (m, 2H) 1.18 (m, 2H) 2.50 (m, 1H) 3.65-3.71 (m, 8H) 5.32 (s, 2H) 6.40 (m, 1H) 7.47 (d, J=8 Hz, 2H) 8.16 (d, J=8 Hz, 2H) 8.52 (s, 1H). MS [M+H] found 408.

### Example 16

### 1-methyl-3-(4-(4′-morpholino-5′H-spiro[cyclopropane-1,7′-furo[3,4-d]pyrimidine]-2′-yl)phenyl)urea

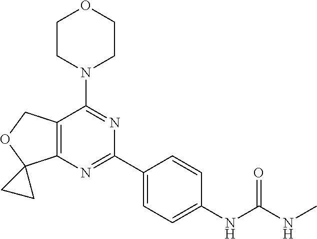

The title compound was prepared by a method similar to Example 14 except 1-methyl-3-(4-(4,4,5,5-tetramethyl-1,3,2-dioxaborolan-2-yl)phenyl)urea was used. ¹H NMR (400 MHz, METHANOL-d6) δ ppm 0.40 (m, 2H) 0.45 (m, 2H) 2.79 (s, 3H) 3.83 (m, 4H) 3.91 (m, 4H) 5.39 (s, 2H) 7.59 (d, J=8 Hz, 2H) 8.06 (d, J=8 Hz, 2H). MS [M+H] found 382.

The compounds of the invention can be administered alone or in the form of a pharmaceutical composition. In practice, the compounds of the invention are usually administered in the form of pharmaceutical compositions, that is, in admixture with pharmaceutically acceptable excipients the proportion and nature of which are determined by the properties of the selected compound of the invention, the chosen route of administration, and standard pharmaceutical practice.

In another embodiment, the present invention provides pharmaceutical compositions comprising: a compound of invention and a pharmaceutically acceptable excipient.

In effecting treatment of a patient in need of such treatment, a compound of the invention can be administered in any form and route which makes the compound bioavailable. The compounds of the invention can be administered by a variety of routes, including oral and parenteral routes, more particularly by inhalation, subcutaneously, intramuscularly, intravenously, transdermally, intranasally, rectally, vaginally, occularly, topically, sublingually, and buccally, intraperitoneally, intravenously, intraarterially, transdermally, sublingually, intramuscularly, rectally, transbuccally, intranasally, intraadiposally, intrathecally and via local delivery for example by catheter or stent.

One skilled in the art can readily select the proper form and route of administration depending upon the particular characteristics of the compound selected, the disorder or condition to be treated, the stage of the disorder or condition, and other relevant circumstances. The pharmaceutical compositions of the invention may be administered to the patient, for example, in the form of tablets, capsules, cachets, papers, lozenges, wafers, elixirs, ointments, transdermal patches, aerosols, inhalants, suppositories, solutions, and suspensions.

The pharmaceutical compositions of the present invention are prepared in a manner well known in the pharmaceutical art and include at least one of the compounds of the invention as the active ingredient. The amount of a compound of the present invention may be varied depending upon its particular form and may conveniently be between 1% to about 70% of the weight of the unit dosage form. The term “pharmaceutically acceptable excipient” refers to those typically used in preparing pharmaceutical compositions and should be pharmaceutically pure and non-toxic in the amounts used. They generally are a solid, semi-solid, or liquid material which can serve as a vehicle or medium for the active ingredient. Some examples of pharmaceutically acceptable excipients are found in Remington's Pharmaceutical Sciences and the Handbook of Pharmaceutical Excipients and include diluents, vehicles, carriers, ointment bases, binders, disintegrates, lubricants, glidants, sweetening agents, flavoring agents, gel bases, sustained release matrices, stabilizing agents, preservatives, solvents, suspending agents, buffers, emulsifiers, dyes, propellants, coating agents, and others.

The present pharmaceutical compositions are preferably formulated in a unit dosage form, each dosage typically containing from about 0.5 mg to about 200 mg of the compounds of the invention. The term “unit dosage form” refers to a physically discrete unit suitable as a single dosage, each unit containing a predetermined quantity of active ingredient, in association with a suitable pharmaceutical excipient, by which one or more is used throughout the dosing regime to produce the desired therapeutic effect.

In one particular variation, the composition is a pharmaceutical composition adapted for oral administration, such as a liquid formulation, for example, a solution or suspension, adapted for oral administration or a tablet or a capsule. In still another particular variation, the pharmaceutical composition is a liquid formulation adapted for parenteral administration.

In another embodiment, the invention provides methods of treating conditions associated with mTOR, comprising: administering to a patient in need thereof an effective amount of a compound of the invention. In another embodiment, the invention provides a method of inhibiting a mTOR: comprising, contacting the enzyme with a compound of the invention. In a further embodiment, the invention provides a method of inhibiting a mTOR: comprising, administering a first compound to a subject that is converted in vivo to a compound of the invention.

In another embodiment, compounds of the invention, including the compound of formula I, are provided for use as a medicament. The invention also provides the use of compounds of the invention, including the use for the manufacture of a medicament, to treat the conditions associated with mTOR described herein. The compounds of the present invention are stable and are relatively safe in their end use. The compounds of the present invention are useful as mTOR inhibitors for a variety of subjects (e.g., humans, non-human mammals and non-mammals).

As used herein terms “condition,” “disorder,” and “disease” relate to any unhealthy or abnormal state. The term “conditions associated with mTOR” includes disorders and diseases in which the inhibition of mTOR provides a therapeutic benefit, such as cancer, allergy/asthma, diseases and conditions of the immune system, inflammation, disease and conditions of the central nervous system (CNS), cardiovascular disease, viral infections, dermatological disease, and diseases and conditions related to uncontrolled angiogenesis, and the like. Where general terms are used herein to describe conditions associated with mTOR it is understood that the more specifically described conditions mentioned in the various diagnostic manuals and other materials are included within the scope of this invention.

For example, it is understood that the treatment of cancer includes treatment of all neoplasia, regardless of their histopathological appearance. Particularly, the cancers that can be treated include, but are not limited to, cancer of blood, including leukemia (including acute myelogenous leukemia, chronic myelogenous leukemia, acute lymphocytic leukemia, chronic lymphocytic leukemia), cancer of the skin, including melanoma, bone, liver, lung (including small-cell lung tumor, non small-cell lung cancer and bronchioalveolar cancer), brain, breast, prostate, larynx, gall bladder, pancreas, rectum, bile duct, parathyroid, thyroid, adrenal, neural tissue, bladder, spleen, head and neck, included the jaw, mouth, and nose, colon, stomach, testes, esophagus, uterus, cervix and vulva, colorectal, bronchi, bile duct, bladder, kidney, ovary, pancreas, multiple myeloma, lymphomas, basal cell carcinoma, squamous cell carcinoma of both ulcerating and papillary type, osteo sarcoma, Ewing's sarcoma, veticulum cell sarcoma, myeloma, giant cell tumor, islet cell tumor, acute and chronic lymphocytic and granulocytic tumors, hairy-cell tumor, adenoma, hyperplasia, medullary carcinoma, pheochromocytoma, mucosal neuronms, intestinal ganglioneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, myelodysplastic syndrome, mycosis fungicide, rhabdomyosarcoma, astrocytoma, non-Hodgkin's lymphoma, Kaposi's sarcoma, osteogenic and other sarcoma, malignant hypercalcemia, polycythermia vera, adenocarcinoma, glioblastoma multiforma, glioma, lymphomas, epidermoid carcinomas, and other carcinomas and sarcomas.

Benign tumors may also be treated by the mTOR inhibitors of the present invention and include, but are not limited to, hemangiomas, hepatocellular adenoma, cavernous haemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas, pyogenic granulomas, and the like, and hamartoma conditions such as Peutz-Jeghers Syndrome (PJS), Cowden disease, Bannayan-Riley-Ruvalcaba Syndrome (BRRS), Proteus syndrome, Lhermitte-Duclos disease and Tuberous Sclerosis (TSC).

The mTOR inhibitors of the present invention may also be used to treat abnormal cell proliferation due to insults to body tissue during surgery. These insults may arise as a result of a variety of surgical procedures such as joint surgery, bowel surgery, and cheloid scarring. Diseases that produce fibrotic tissue include emphysema. Repetitive motion disorders that may be treated using the present invention include carpal tunnel syndrome.

The mTOR inhibitors of the invention may also be useful in the prevention of restenosis, that is the control of undesired proliferation of normal cells in the vasculature in response to the introduction of stents in the treatment of vasculature disease.

Proliferative responses associated with organ transplantation that may be treated using mTOR inhibitors of the invention include proliferative responses contributing to potential organ rejections or associated complications. Specifically, these proliferative responses may occur during transplantation of the heart, lung, liver, kidney, and other body organs or organ systems.

The mTOR inhibitors of the invention may also be useful the treatment of abnormal angiogenesis including the abnormal angiogenesis accompanying rheumatoid arthritis, ischemic-reperfusion related brain edema and injury, cortical ischemia, ovarian hyperplasia and hypervascularity, (polycystic ovary syndrom), endometriosis, psoriasis, diabetic retinopaphy, and other ocular angiogenic diseases such as retinopathy of prematurity (retrolental fibroplastic), macular degeneration, corneal graft rejection, neuroscular glaucoma, Oster Webber syndrome, retinal/choroidal neuvascularization and corneal neovascularization, Best's disease, myopia, optic pits, Stargart's diseases, Pagets disease, vein occlusion, artery occlusion, sickle cell anemia, sarcoid, syphilis, pseudoxanthoma elasticum carotid abostructive diseases, chronic uveitis/vitritis, mycobacterial infections, Lyme's disease, systemic lupus erythematosis, retinopathy of prematurity, Eales disease, diabetic retinopathy, macular degeneration, Bechets diseases, infections causing a retinitis or chroiditis, presumed ocular histoplasmosis, pars planitis, chronic retinal detachment, hyperviscosity syndromes, toxoplasmosis, trauma and post-laser complications, diseases associated with rubesis (neovascularization of the angle), diseases caused by the abnormal proliferation of fibrovascular or fibrous tissue including all forms of proliferative vitreoretinopathy, atopic keratitis, superior limbic keratitis, pterygium keratitis sicca, sjogrens, acne rosacea, phylectenulosis, diabetic retinopathy, retinopathy of prematurity, corneal graft rejection, Mooren's ulcer, Terrien's marginal degeneration, marginal keratolysis, polyarteritis, Wegener sarcoidosis, scleritis, periphigoid radial keratotomy, neovascular glaucoma and retrolental fibroplasia, syphilis, Mycobacteria infections, lipid degeneration, chemical burns, bacterial ulcers, fungal ulcers, Herpes simplex infections, Herpes zoster infections, protozoan infections, and Kaposi sarcoma, Alzheimer's disease, Parkinson's disease amyotrophic lateral sclerosis (ALS), epilepsy, seizures, Huntington's disease, polyglutamine diseases, traumatic brain injury, ischemic and hemorrhaging stroke, cerebral ischemias or neurodegenerative disease, including apoptosis-driven neurodegenerative disease, caused by traumatic injury, acute hypoxia, ischemia or glutamate neurotoxicity.

For example, it is understood that the treatment of inflammation include, but are not limited to, acute pancreatitis, chronic pancreatitis, asthma, allergies, chronic obstructive pulmonary disease, adult respiratory distress syndrome. and chronic inflammatory diseases associated with uncontrolled angiogenesis, inflammatory bowel diseases such as Crohn's disease and ulcerative colitis, psoriasis, sarcoidois, and rheumatoid arthritis. sarcoidosis, multisystem granulomatous disorder.

For example, it is understood that the treatment of autoimmune includes, but are not limited to, glomerulonephritis, rheumatoid arthritis, systemic lupus erythematosus, scleroderma, chronic thyroiditis, Graves' disease, autoimmune gastritis, diabetes, autoimmune hemolytic anemia, autoimmune neutropenia, thrombocytopenia, atopic dermatitis, chronic active hepatitis, myasthenia gravis, multiple sclerosis, inflammatory bowel disease, ulcerative colitis, Crohn's disease, psoriasis, graft vs. host disease, multiple sclerosis, or Sjoegren's syndrome.

A wide variety of therapeutic agents may have a therapeutic additive or synergistic effect with mTOR inhibitors according to the present invention. Combination therapies that comprise one or more compounds of the present invention with one or more other therapeutic agents can be used, for example, to: 1) enhance the therapeutic effect(s) of the one or more compounds of the present invention and/or the one or more other therapeutic agents; 2) reduce the side effects exhibited by the one or more compounds of the present invention and/or the one or more other therapeutic agents; and/or 3) reduce the effective dose of the one or more compounds of the present invention and/or the one or more other therapeutic agents. It is noted that combination therapy is intended to cover when agents are administered before or after each other (sequential therapy) as well as when the agents are administered at the same time.

Examples of such therapeutic agents that may be used in combination with the present mTOR inhibitors include, but are not limited to, anti-cell proliferation agents, anticancer agents, alkylating agents, antibiotic agents, antimetabolic agents, hormonal agents, plant-derived agents, and biologic agents.

Examples of such therapeutic agents that may be used in combination with mTOR inhibitors include, but are not limited to, anti-cell proliferation agents, anticancer agents, alkylating agents, antibiotic agents, antimetabolic agents, hormonal agents, plant-derived agents, and biologic agents.

Anti-cell proliferation agents useful in combination with the mTOR inhibitors of the present invention include, but are not limited to, retinoid acid and derivatives thereof, 2-methoxyestradiol, ANGIOSTATIN™ protein, ENDOSTATIN™ protein, suramin, squalamine, tissue inhibitor of metalloproteinase-I, tissue inhibitor of metalloproteinase-2, plasminogen activator inhibitor-1, plasminogen activator inhibitor-2, cartilage-derived inhibitor, paclitaxel, platelet factor 4, protamine sulphate (clupeine), sulphated chitin derivatives (prepared from queen crab shells), sulphated polysaccharide peptidoglycan complex (sp-pg), staurosporine, modulators of matrix metabolism, including for example, proline analogs ((1-azetidine-2-carboxylic acid (LACA), cishydroxyproline, d,1-3,4-dehydroproline, thiaproline, beta-aminopropionitrile fumarate, 4-propyl-5-(4-pyridinyl)-2(3H)-oxazolone, methotrexate, mitoxantrone, heparin, interferons, 2 macroglobulin-serum, chimp-3, chymostatin, beta.-cyclodextrin tetradecasulfate, eponemycin; fumagillin, gold sodium thiomalate, d-penicillamine (CDPT), beta-1-anticollagenase-serum, alpha-2-antiplasmin, bisantrene, lobenzarit disodium, n-(2-carboxyphenyl-4-chloroanthronilic acid disodium or “CCA”, thalidomide, angostatic steroid, cargboxynaminolmidazole, metalloproteinase inhibitors such as BB94. Other anti-angiogenesis agents that may be used include antibodies, preferably monoclonal antibodies against these angiogenic growth factors: bFGF, aFGF, FGF-5, VEGF isoforms, VEGF-C, HGF/SF and Ang-1/Ang-2.

Alkylating agents useful in combination with the present mTOR inhibitors include, but are not limited to, bischloroethylamines (nitrogen mustards, e.g. chlorambucil, cyclophosphamide, ifosfamide, mechlorethamine, melphalan, uracil mustard), aziridines (e.g. thiotepa), alkyl alkone sulfonates (e.g. busulfan), nitrosoureas (e.g. carmustine, lomustine, streptozocin), nonclassic alkylating agents (altretamine, dacarbazine, and procarbazine), platinum compounds (carboplastin and cisplatin). Combination therapy including a mTOR inhibitor and an alkylating agent is expected to have therapeutic synergistic effects in the treatment of cancer and reduce sides affects associated with these chemotherapeutic agents.

Examples of antibiotic agents useful in combination with the present mTOR inhibitors include, but are not limited to, anthracyclines (e.g. doxorubicin, daunorubicin, epirubicin, idarubicin and anthracenedione), mitomycin C, bleomycin, dactinomycin, plicatomycin. These antibiotic agents interfere with cell growth by targeting different cellular components.

Antimetabolic agents useful in combination with the present mTOR inhibitors include, but are not limited to, fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, and gemcitabine. Combination therapy including a mTOR inhibitor and an antimetabolic agent is expected to have therapeutic synergistic effects on cancer and reduce sides affects associated with these chemotherapeutic agents.

Hormonal agents useful in combination with the present mTOR inhibitors include synthetic estrogens (e.g. diethylstibestrol), antiestrogens (e.g. tamoxifen, toremifene, fluoxymesterol and raloxifene), antiandrogens (bicalutamide, nilutamide, flutamide), aromatase inhibitors (e.g., aminoglutethimide, anastrozole and tetrazole), ketoconazole, goserelin acetate, leuprolide, megestrol acetate and mifepristone. Combination therapy including a mTOR inhibitor and a hormonal agent is expected to have therapeutic synergistic effects on cancer and reduce sides affects associated with these chemotherapeutic agents.

Plant-derived agents useful in combination with the present mTOR inhibitors include, but are not limited to, vinca alkaloids (e.g., vincristine, vinblastine, vindesine, vinzolidine and vinorelbine), podophyllotoxins (e.g., etoposide (VP-16) and teniposide (VM-26)), taxanes (e.g., paclitaxel and docetaxel). These plant-derived agents generally act as antimitotic agents that bind to tubulin and inhibit mitosis. Podophyllotoxins such as etoposide are believed to interfere with DNA synthesis by interacting with topoisomerase II, leading to DNA strand scission. Combination therapy including an mTOR inhibitor and a plant-derived agent is expected to have therapeutic synergistic effects on cancer and reduce sides affects associated with these chemotherapeutic agents

The terms “treat,” “treatment,” and “treating” include improvement of the conditions described herein. Also, it is also recognized that one skilled in the art may affect the conditions by treating a patient presently afflicted with the disorders or by prophylactically treating a patient believed to be susceptible to such conditions with an effective amount of a compound of invention. Thus, the terms “treat,” “treatment,” and “treating” include all processes providing slowing, interrupting, arresting, controlling, or stopping of the state or progression of the conditions described herein, but does not necessarily indicate a total elimination of all symptoms or a cure of the condition, and is intended to include prophylactic and therapeutic treatment of such disorders.

As used herein the terms “patient” and “subject” includes humans and non-human animals, for example, mammals, such as mice, rats, guinea pigs, dogs, cats, rabbits, cows, horses, sheep, goats, and pigs. The term also includes birds, fish, reptiles, amphibians, and the like. It is understood that a more particular patient is a human. Also, more particular patients and subjects are non-human mammals, such as mice, rats, and dogs.

As used herein, the term “effective amount” refers to the amount of compound of the invention which treats, upon single or multiple dose administration, a patient suffering from the mentioned condition. An effective amount can be readily determined by the attending diagnostician, as one skilled in the art, by the use of known techniques and by observing results obtained under analogous circumstances. In determining the effective amount, the dose, a number of factors are considered by the attending diagnostician, including, but not limited to: the species of patient; its size, age, and general health; the specific condition, disorder, or disease involved; the degree of or involvement or the severity of the condition, disorder, or disease, the response of the individual patient; the particular compound administered; the mode of administration; the bioavailability characteristics of the preparation administered; the dose regimen selected; the use of concomitant medication; and other relevant circumstances. An effective amount of the present use invention, including a compound of the invention, is expected to vary from about 0.1 milligram per kilogram of body weight per day (mg/kg/day) to about 20 mg/kg/day. Specific amounts can be determined by the skilled person.

In a particular embodiment the present invention provides a method for treating cancer, comprising: administering to a patient in need thereof an effective amount of a compound of invention.

The invention also provides an article of manufacture: comprising at least one compound of the invention and a label. The label may include information about the manufacturer, doses, conditions to be treated, and the use of the compound or pharmaceutical composition.

In another embodiment the invention provides a kit: comprising, at least one compound of the invention, a label, and apparatus for administration. The apparatus may include mixing vials, liquids for forming solutions or suspensions, tubing, syringes, and the like.

The activity of compounds as mTOR inhibitors may be determined by a variety of methods, including in vitro and in vivo methods.

Example A Inhibition of mTOR

Purified mTor are purchased from Invitrogen. mTor activity was determined using Invitrogen's LanthaScreen system. The inhibitory properties of compounds relative to mTor may be determined using a black 384-well-plate format in the following buffer 50 mM Hepes, 10 mM NaCl, 10 mM MgCl₂, 0.2 mM EDTA, 0.01% Brij35, 2 mM DTT at pH7.3. The test compound is prepared in DMSO using 2 fold serial dilutions for 11 data points which are added to the buffer so that each dilution contains 3% DMSO.

An assay for mTor inhibition is as follows:

Combine in each well 2 μl of 1.2 μM GFP-4E-BP1 (Invitrogen) and 150 μM ATP (in buffer), 2 μl of diluted test compound (3% DMSO in buffer), and 2 μl of 6 nM mTor in buffer. The reaction mixture is then incubated at room temperature for 30 min, and quenched by adding 40 mM ETDA with 4 nM Tb-anti-p4E-BP1 [pThr46] antibody in TR-FRET dilution buffer (Invitrogen). The plate is kept at room temperature for 1 hour and then read using PheraStar (BMG labtech) LanthaScreen mode.

pIC₅₀ values, the negative of the log of the IC₅₀, are calculated by non-linear curve fitting of the compound concentrations and percent of inhibition to the standard pIC₅₀ equation. The exemplified compounds inhibited human mTOR in the assay of Example A with a pIC₅₀ of: A less than about 6, B between 6 and 7.5, and C greater than 7.5 as indicated in Table 1.

TABLE 1

| | | | | | | | |
|---|---|---|---|---|---|---|---|
| 2 | C | 4 | C | 6 | C | 8 | C |
| 9 | C | 10 | C | 11 | C | 12 | B |
| 13 | C | 14 | C | 15 | C | 16 | C |

What is claimed is:

1. A compound of the formula 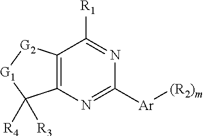
Ar is selected from the group consisting of C₄₋₁₄ aryl, and C₁₋₁₀ heteroaryl;
R₁ is selected from the group consisting of optionally substituted C₃₋₈ cycloalkyl, and optionally substituted C₃₋₁₂ heterocycloalkyl;
R₂ is, each time taken, independently selected from the group consisting of halo, cyano, optionally substituted C₁₋₆ alkyl, C₁₋₈ sulfonyl, optionally substituted C₂₋₄ alkenyl, optionally substituted C₂₋₄ alkynyl, optionally substituted C₁₋₄ alkoxy, C₀₋₈ alkylamino, optionally substituted C₄₋₁₄ aryl, optionally substituted C₄₋₁₄ aryloxy, C₁₋₅ oxycarbonyl, C₁₋₅ carbonyloxy, optionally substituted C₃₋₆ heterocycloalkyl, optionally substituted C₁₋₁₀ heteroaryl, hydroxy, nitro, —NHC(O)NR₇R₈, —NHC(O)OR₉, —NH(SO₂)NHR₇, —NHC(O)NHNR₇R₈, —NHC(S)NR₇R₈, —NHC(═NR₁₀)NR₇R₈, —NHC(SR₁₁)NR₇R₈, and —NHC(═NR₁₀)OR₁₂;
R₃ is selected from the group consisting of halo, optionally substituted C₁₋₆ alkyl, C₁₋₈ sulfonyl, optionally substituted C₂₋₄ alkenyl, optionally substituted C₁₋₄ alkoxy, and optionally substituted C₃₋₈ cycloalkyl;
R₄ is selected from the group consisting of halo, optionally substituted C₁₋₆ alkyl, C₁₋₈ sulfonyl, optionally substituted C₂₋₄ alkenyl, optionally substituted C₁₋₄ alkoxy, and optionally substituted C₃₋₈ cycloalkyl; or
R₃ and R₄ are taken together along with the carbon to which they are attached to form a spiro mono- or bi-cyclic ring having 3 to 10 carbon atoms and optionally having 1, 2, or 3 heteroatoms independently selected from the group consisting of oxygen, nitrogen and sulfur and optionally oxidized on sulfur to provide the sulfoxides and sulfone and optionally substituted on the ring carbons with 1 to 4 substituents, each time taken, independently selected from the group consisting of optionally substituted C₁₋₄ alkyl, C₂₋₄ alkenyl, C₁₋₄ alkoxy, C₁₋₉ amide, C₁₋₇ amido, C₀₋₈ alkylamino, C₁₋₅ oxycarbonyl, cyano, C₃₋₈ cycloalkyl, C₃₋₈ cycloalkoxy, halo, hydroxy, nitro, oxo, and optionally substituted phenyl, and optionally substituted on the ring nitrogens, each time taken, with a substituent selected from the group consisting of optionally substituted C₁₋₄ alkyl, C₂₋₄ alkenyl, C₃₋₈ cycloalkyl, optionally substituted C₃₋₆ heterocycloalkyl, and optionally substituted phenyl;
G₁ is selected from the group consisting of O and CR₅R₆;
G₂ is selected from the group consisting of O and CR₅R₆;
provided that one of G₁ or G₂ is O and the other is CR₅R₆;
R₅ is selected from the group consisting of hydrogen, halo, optionally substituted C₁₋₆ alkyl, optionally substituted C₂₋₄ alkenyl, and optionally substituted C₃₋₈ cycloalkyl;
R₆ is selected from the group consisting of hydrogen, halo, optionally substituted C₁₋₆ alkyl, optionally substituted C₂₋₄ alkenyl, and optionally substituted C₃₋₈ cycloalkyl;
R₇ is, each time taken, independently selected from the group consisting of hydrogen, optionally substituted C₁₋₆ alkyl, optionally substituted C₃₋₈ cycloalkyl, optionally substituted C₄₋₁₄ aryl, optionally substituted C₃₋₆ heterocycloalkyl, and optionally substituted C₁₋₁₀ heteroaryl;
R₈ is, each time taken, independently selected from the group consisting of hydrogen, optionally substituted C₁₋₆ alkyl, optionally substituted C₃₋₈ cycloalkyl, optionally substituted C₄₋₁₄ aryl, optionally substituted C₃₋₆ heterocycloalkyl, and optionally substituted C₁₋₁₀ heteroaryl;
R₉ is, each time taken, independently selected from the group consisting of optionally substituted C₁₋₆ alkyl, optionally substituted C₃₋₈ cycloalkyl, optionally substituted C₄₋₁₄ aryl, and optionally substituted C₃₋₆ heterocycloalkyl;
R₁₀ is, each time taken, independently selected from the group consisting of hydrogen, optionally substituted C₁₋₆ alkyl, optionally substituted C₁₋₆ alkoxy, optionally substituted C₃₋₈ cycloalkyl, optionally substituted C₄₋₁₄ aryl, optionally substituted C₃₋₆ heterocycloalkyl, optionally substituted C₁₋₁₀ heteroaryl, cyano, and nitro;
R₁₁ is each time taken independently selected from the group consisting of optionally substituted C₁₋₆ alkyl and optionally substituted phenyl;
R₁₂ is each time taken independently selected from the group consisting of optionally substituted C₁₋₆ alkyl, optionally substituted C₃₋₈ cycloalkyl, and optionally substituted C₄₋₁₄ aryl;
m is 0, 1, 2, 3, and 4;
or the pharmaceutically acceptable salts thereof.

2. A compound of claim 1 wherein Ar is C₄₋₁₄ aryl.

3. A compound of claim 2 wherein Ar is phenyl.

4. A compound of any one of claims 1 to 3 wherein m is 1 or 2.

5. A compound of any one of claims 1 to 4 wherein R₃ is C₁₋₆ alkyl.

6. A compound of any one of claims 1 to 5 wherein R₄ is C₁₋₆ alkyl.

7. A compound of any one of claims 1 to 6 wherein G₁ is O and G₂ is CR₅R₆.

8. A compound of any one of claims 1 to 7 wherein R₁ is selected from the group consisting of N-morpholinyl and N-8-oxa-3-azabicyclo[3.2.1]octanyl.

9. A compound of any one of claims 1 to 8 wherein m is 1 and R₂, is —NHC(O)NR₇R₈.

10. A compound of claim 1 selected from the group consisting of 2-(4-bromophenyl)-7,7-dimethyl-4-morpholino-6,7-dihydrofuro[3,2-d]pyrimidine, 1-(4-(7,7-dimethyl-4-morpholino-6,7-dihydrofuro[3,2-d]pyrimidin-2-yl)phenyl)-3-methylurea, 4-(8-oxa-3-azabicyclo[3.2.1]octan-3-yl)-2-(4-bromophenyl)-7,7-dimethyl-6,7-dihydrofuro[3,2-d]pyrimidine, 1-(4-(4-(8-oxa-3-azabicyclo[3.2.1]octan-3-yl)-7,7-dimethyl-6,7-dihydrofuro[3,2-d]pyrimidin-2-yl)phenyl)-3-methylurea, 4-(8-oxa-3-azabicyclo[3.2.1]octan-3-yl)-2-(4-bromophenyl)-7,7-dimethyl-5,7-dihydrofuro[3,4-d]pyrimidine, 1-(4-(4-(8-oxa-3-azabicyclo[3.2.1]octan-3-yl)-7,7-dimethyl-5,7-dihydrofuro[3,4-d]pyrimidin-2-yl)phenyl)-3-methylurea, 2-(4-bromophenyl)-7,7-dimethyl-4-morpholino-5,7-dihydrofuro[3,4-d]pyrimidine, 1-(4-(7,7-dimethyl-4-morpholino-5,7-dihydrofuro[3,4-d]pyrimidin-2-yl)phenyl)-3-methylurea, 1-(4-(4-(8-oxa-3-azabicyclo[3.2.1]octan-3-yl)-7,7-dimethyl-5,7-dihydrofuro[3,4-d]pyrimidin-2-yl)phenyl)-3-ethylurea, 1-(4-(7,7-dimethyl-4-morpholino-5,7-dihydrofuro[3,4-d]pyrimidin-2-yl)phenyl)-3-ethylurea, 1-cyclopropyl-3-(4-(7,7-dimethyl-4-morpholino-5,7-dihydrofuro[3,4-d]pyrimidin-2-yl)phenyl)urea, 1-(4-(4-(8-oxa-3-azabicyclo[3.2.1]octan-3-yl)-7-hydroxy-7-methyl-5,7-dihydrofuro[3,4-d]pyrimidin-2-yl)phenyl)-3-methylurea, 1-(4-(4′-(8-oxa-3-azabicyclo[3.2.1]octan-3-yl)-5′H-spiro[cyclopropane-1,7′-furo[3,4-d]pyrimidine]-2′-yl)phenyl)-3-methylurea, 1-ethyl-3-(4-(4′-morpholino-5′H-spiro[cyclopropane-1,7′-furo[3,4-d]pyrimidine]-2′-yl)phenyl)urea, 1-cyclopropyl-3-(4-(4′-morpholino-5′H-spiro[cyclopropane-1,7′-furo[3,4-d]pyrimidine]-2′-yl)phenyl)urea, and 1-methyl-3-(4-(4′-morpholino-5′H-spiro[cyclopropane-1,7′-furo[3,4-d]pyrimidine]-2′-yl)phenyl)urea.

11. A pharmaceutical composition, comprising: a compound of claim 1 and a pharmaceutically acceptable excipient.

12. The use of a compound of claim 1 as a medicament.

13. The use of a compound of any one of claims 1 to 9 for the manufacture of a medicament for treating conditions associated with mTOR.

14. A method of treating conditions associated with mTOR, comprising: administering to a patient in need thereof an effective amount of a compound of claim 1.
